(19) United States
(12) Patent Application Publication
(10) Pub. No.: US 2018/0098690 A1
(43) Pub. Date: Apr. 12, 2018

(54) ENDOSCOPE APPARATUS AND METHOD FOR OPERATING ENDOSCOPE APPARATUS

(71) Applicant: OLYMPUS CORPORATION, Tokyo (JP)

(72) Inventor: Hidekazu IWAKI, Tokyo (JP)

(73) Assignee: OLYMPUS CORPORATION, Tokyo (JP)

(21) Appl. No.: 15/836,235

(22) Filed: Dec. 8, 2017

Related U.S. Application Data
(63) Continuation of application No. PCT/JP2015/066887, filed on Jun. 11, 2015.

(51) Int. Cl. A61B 1/05; A61B 1/00; G02B 23/24; G06T 7/00

(52) U.S. Cl. CPC A61B 1/05; A61B 1/00188; A61B 1/00186; G02B 23/2407; G06T 7/0012; A61B 1/00009

(57) ABSTRACT

An endoscope apparatus includes a processor including hardware, the processor being configured to implement: an image acquisition process, an attention region detection process, a motion vector estimation process, and a display control process that displays an alert image based on an attention region and a motion vector. The processor implements display control process that performs display control on the alert image in the second captured image to achieve the second object region that is smaller than the first object region.

### CROSS REFERENCE TO RELATED APPLICATION

This application is a continuation of International Patent Application No. PCT/JP2015/066887, having an international filing date of Jun. 11, 2015, which designated the United States, the entirety of which is incorporated herein by reference.

### BACKGROUND

In some cases, observation using an endoscope may be performed with information related to an attention region, such as a result of lesion detection from a system, presented based on a result of image analysis. In some conventional cases, such information from the system has been presented while being overlaid at a predetermined position relative to the attention region on an observation screen, through a predetermined method. The information thus presented in an overlaid manner could be in the way of observation in some cases. Thus, various methods have been developed for such a type of presentation to display information without interfering with the observation.

For example, JP-A-2011-255006 discloses a method of removing information that has been presented, when at least one of the number of attention regions, the size of the regions, and a period that has elapsed after the first detection exceeds a predetermined threshold value.

JP-A-2011-087793 discloses a method of overlaying a mark (image data) indicating the position of a lesion part of an attention region selected with a selection unit.

JP-A-2001-104333 discloses a method in which the size, a displayed location, and displaying/hiding of an overlaid window can be changed.

JP-A-2009-226072 discloses a method in which when an image is determined to have changed, shifted amounts of the image at various portions are calculated, and information to be overlaid is changed in accordance with the shifted amounts thus calculated.

### SUMMARY

According to one aspect of the invention, there is provided an endoscope apparatus comprising:

a processor comprising hardware,

the processor being configured to implement:

an image acquisition process that acquires a captured image, the captured image being an image of an object obtained by an imaging section;

an attention region detection process that detects an attention region based on a feature quantity of pixels in the captured image;

a motion vector estimation process that estimates a motion vector in at least a part of the captured image; and

a display control process that displays an alert image on the captured image in an overlaid manner based on the attention region and the motion vector, the alert image highlighting the attention region,

wherein a first image region is defined as an region, in a first captured image, where the alert image is overlaid on the attention region, and a first object region is defined as an region, on the object, corresponding to the first image region,

wherein a second image region is defined as an region, in a second captured image, where the alert image is overlaid on an image region corresponding to the first object region, and a second object region is defined as an region, on the object, corresponding to the second image region, and

wherein the processor implements the display control process that performs display control on the alert image in the second captured image to achieve the second object region that is smaller than the first object region.

According to another aspect of the invention, there is provided a method for operating an endoscope apparatus comprising:

performing processing to acquire a captured image, the captured image being an image of an object obtained by an imaging section;

detecting an attention region based on a feature quantity of pixels in the captured image;

estimating a motion vector in at least a part of the captured image; and

performing display control to display an alert image on the captured image in an overlaid manner based on the attention region and the motion vector, the alert image highlighting the attention region,

wherein a first image region is defined as an region, in a first captured image, where the alert image is overlaid on the attention region, and a first object region is defined as an region, on the object, corresponding to the first image region,

wherein a second image region is defined as an region, in a second captured image, where the alert image is overlaid on an image region corresponding to the first object region, and a second object region is defined as an region, on the object, corresponding to the second image region, and

wherein in the display control, display control is performed on the alert image in the second captured image to achieve the second object region that is smaller than the first object region.

### BRIEF DESCRIPTION OF THE DRAWINGS

FIG. 1 illustrates a relationship between an attention region and an alert image.

FIG. 2 illustrates an example of a configuration of an endoscope apparatus.

FIG. 3A to FIG. 3D illustrate a first image region and a second image region in a case where translational motion occurs.

FIG. 4A and FIG. 4B illustrate the first image region and an region on the second captured image corresponding to the first image region in a case where zoom-in occurs.

FIG. 5 illustrates a configuration example of the endoscope apparatus in detail.

FIG. 6A and FIG. 6B illustrate a method of hiding the alert image in a case where zoom-in occurs.

FIG. 7A and FIG. 7B illustrate a method of hiding the alert image in a case where a translational motion toward an image center portion occurs.

FIG. 8A to FIG. 8E illustrate a method of rotating the alert image.

FIG. 9A and FIG. 9B illustrate a method of rotating an alert image for displaying character information.

FIG. 10 illustrates a method of setting a rotation amount of the alert image based on a size of the motion vector.

FIG. 11A to FIG. 11C illustrate a method of changing a shape of the alert image based on a pan/tilt operation.

FIG. 12 illustrates a method of simply changing a shape of the alert image based on a pan/tilt operation.

FIG. 13A to FIG. 13C illustrate a method of reducing a size of the alert image in a case where zoom-in occurs.

FIG. 14A and FIG. 14B illustrate a method of displaying a plurality of alert images for an attention region, and a method of causing the alert images to make a translational motion based on a motion vector.

FIG. 15A to FIG. 15C illustrates multi-stage display control.

### DESCRIPTION OF EXEMPLARY EMBODIMENTS

According to one embodiment of the invention, there is provided an endoscope apparatus comprising:

a processor comprising hardware,

the processor being configured to implement:

an image acquisition process that acquires a captured image, the captured image being an image of an object obtained by an imaging section;

an attention region detection process that detects an attention region based on a feature quantity of pixels in the captured image;

a motion vector estimation process that estimates a motion vector in at least a part of the captured image; and

a display control process that displays an alert image on the captured image in an overlaid manner based on the attention region and the motion vector, the alert image highlighting the attention region,

wherein a first image region is defined as an region, in a first captured image, where the alert image is overlaid on the attention region, and a first object region is defined as an region, on the object, corresponding to the first image region,

wherein a second image region is defined as an region, in a second captured image, where the alert image is overlaid on an image region corresponding to the first object region, and a second object region is defined as an region, on the object, corresponding to the second image region, and

wherein the processor implements the display control process that performs display control on the alert image in the second captured image to achieve the second object region that is smaller than the first object region.

According to another embodiment of the invention, there is provided a method for operating an endoscope apparatus comprising:

performing processing to acquire a captured image, the captured image being an image of an object obtained by an imaging section;

detecting an attention region based on a feature quantity of pixels in the captured image;

estimating a motion vector in at least a part of the captured image; and

performing display control to display an alert image on the captured image in an overlaid manner based on the attention region and the motion vector, the alert image highlighting the attention region,

wherein a first image region is defined as an region, in a first captured image, where the alert image is overlaid on the attention region, and a first object region is defined as an region, on the object, corresponding to the first image region,

wherein a second image region is defined as an region, in a second captured image, where the alert image is overlaid on an image region corresponding to the first object region, and a second object region is defined as an region, on the object, corresponding to the second image region, and

wherein in the display control, display control is performed on the alert image in the second captured image to achieve the second object region that is smaller than the first object region.

The exemplary embodiments of the invention are described below. Note that the following exemplary embodiments do not in any way limit the scope of the invention laid out in the claims. Note also that all of the elements described below in connection with the exemplary embodiments should not necessarily be taken as essential elements of the invention.

### 1. Method According to the Present Embodiment

First of all, a method according to the present embodiment is described. One conventionally known method includes: detecting an attention region in a captured image obtained with an endoscope; and displaying the attention region provided with predetermined information. For example, with endoscopy, a physician makes a diagnosis while viewing an endoscope image, to check whether a body cavity of an examinee includes any abnormal portion. Unfortunately, such a visual diagnosis involves a risk of overlooking lesion parts such as a small lesion and a lesion similar to a peripheral portion.

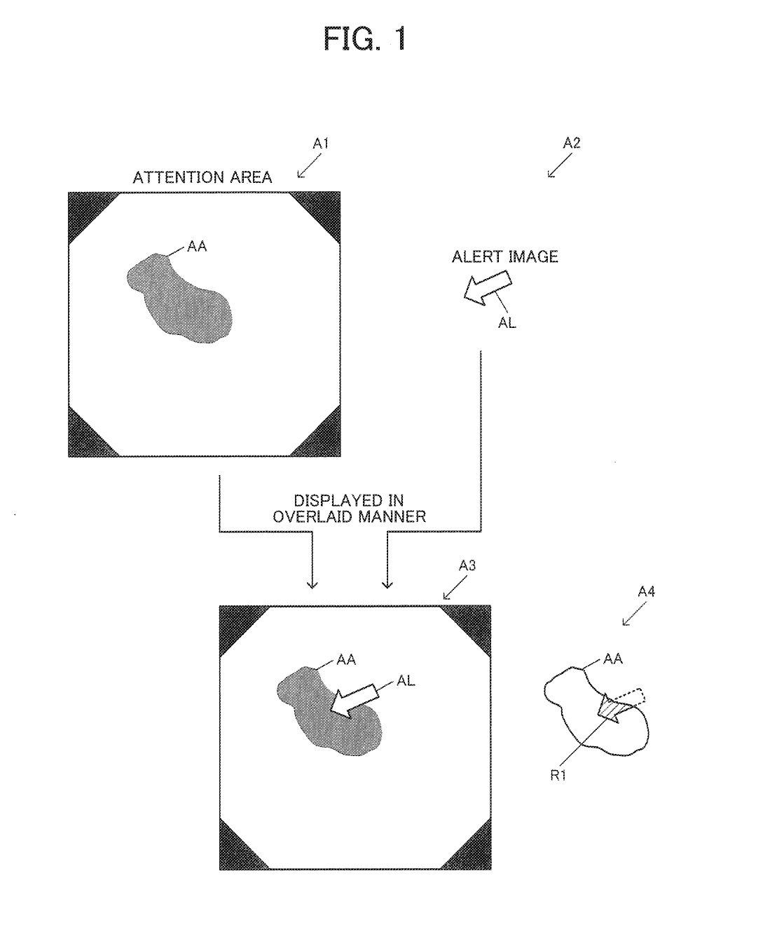

Thus, an region that may include a lesion is detected as an attention region AA, in a captured image, as illustrated in a section A1 in FIG. 1. Then, an alert image AL (an arrow in this example) is displayed on the region as illustrated in a section A2 in FIG. 1. Thus, a physician can be prevented from overlooking the lesion, and a smaller work load on the physician can be achieved. More specifically, as illustrated in a section A3 in FIG. 1, a method of displaying the arrow (in a wide sense, the alert image AL), indicating the position of the attention region AA, at a position corresponding to the attention region may be employed. With such a method, information indicating that the attention region has been detected and indicating the position of the detected attention region on the captured image can be presented in a clearly recognizable manner to a user viewing the image. Information indicating more than the position can be presented by using an alert image including characters and the like. The endoscope apparatus according to the present embodiment may be a medical endoscope apparatus in a narrow sense. A description is given below with the medical endoscope apparatus as an example.

Unfortunately, the alert image displayed on the captured image hinders the observation of an object underlying the alert image. For example, an opaque alert image makes an underlying object visually not recognizable in the captured image. In particular, as illustrated in the section A3 in FIG. 1 in which the alert image AL is overlaid on the attention region AA, observation of the attention region AA, including a captured image of an object of interest, in an overlaid region is inevitably hindered. Specifically, the overlaid region corresponds to an region R1 in the attention region AA illustrated in a section A4 in FIG. 1.

In view of this, JP-A-2011-255006, JP-A-2011-087793, JP-A-2001-104333, and JP-A-2009-226072 and the like disclose conventional methods for controlling information displayed on a captured image. However, the conventional methods require a predetermined condition to be satisfied or require a predetermined operation to be performed, for hiding the alert image. For example, the condition that needs to be satisfied for removing the alert image may include: the number of attention regions and the size of the regions exceeding a predetermined threshold value; and a period that has elapsed after detection of the attention region exceeding a predetermined threshold value. In such a case, a user needs to be aware of the condition, and somehow increase the number or the attention regions or the size of the regions or wait for elapse of the predetermined period. Furthermore, the user might even have to go through a cumbersome operation for controlling the alert image. Examples of such an operation include selecting an attention region or an alert region and setting a display mode.

JP-A-2009-226072 discloses a method of changing displayed information based on movement on an image, that is, relative movement between an imaging section and an object. This method enables an alert image to be changed without a special operation. However, the method disclosed in JP-A-2009-226072 is not directed to the improvement of the observation condition compromised by the alert image. Thus, the change in the information does not necessarily result in an improved observation condition of the attention region. In other words, the method for changing the information (alert image) disclosed is not for improving the observation condition of the attention region.

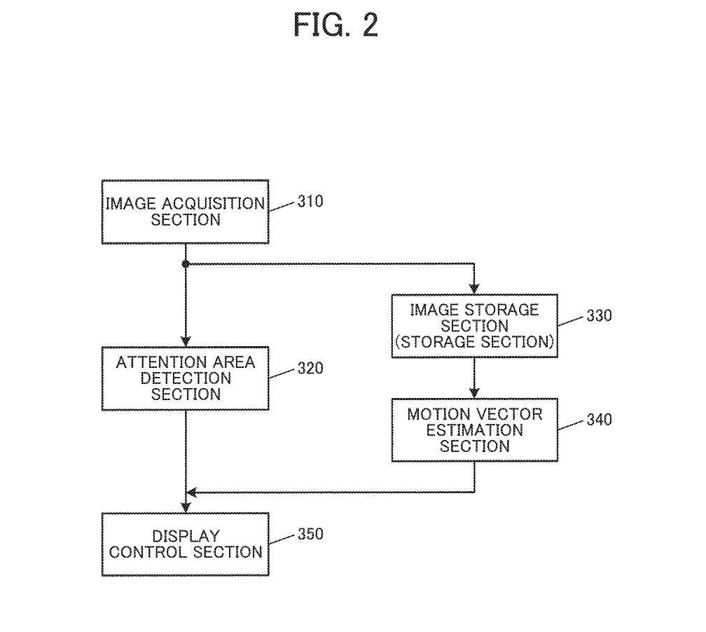

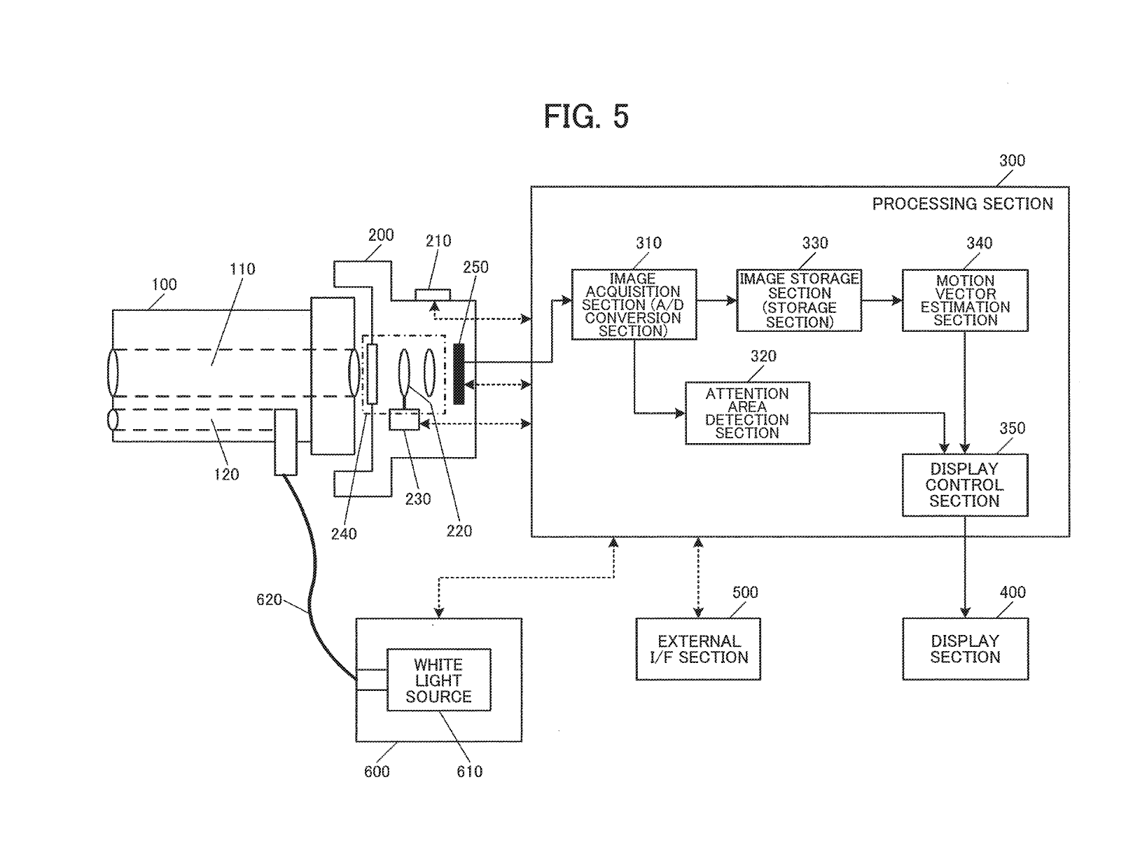

In view of the above, the applicant proposes a method of controlling a display mode of an alert image to improve the observation condition of the attention region, without a cumbersome operation by a user or the like. More specifically, as illustrated in FIG. 2, an endoscope apparatus according to the present embodiment includes: an image acquisition section 310 that acquires a captured image obtained by capturing an image of an object with an imaging section (for example, an imaging section 200 in FIG. 5 described below); an attention region detection section 320 that detects an attention region based on a feature quantity of pixels in the captured image; a motion vector estimation section 340 that estimates a motion vector in at least a part of the captured image; and a display control section 350 that displays an alert image, highlighting the attention region, on the captured image in an overlaid manner based on the attention region and the motion vector. An region, in a first captured image, where the alert image is overlaid on the attention region is referred to as a first image region. An region, on the object, corresponding to the first image region is referred to as a first object region. An region, in a second captured image, where the alert image is overlaid on an image region corresponding to the first object region is referred to as a second image region. An region, on the object, corresponding to the second image region is referred to as a second object region. The display control section 350 performs display control on the alert image in the second captured image, to achieve the second object region that is smaller than the first object region.

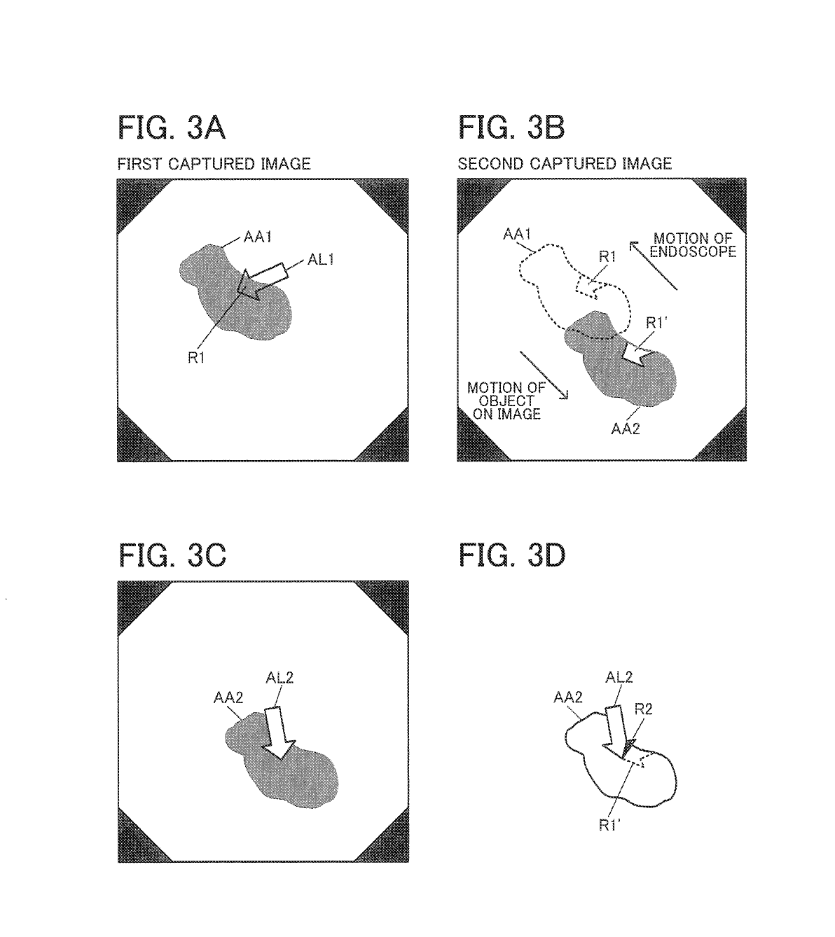

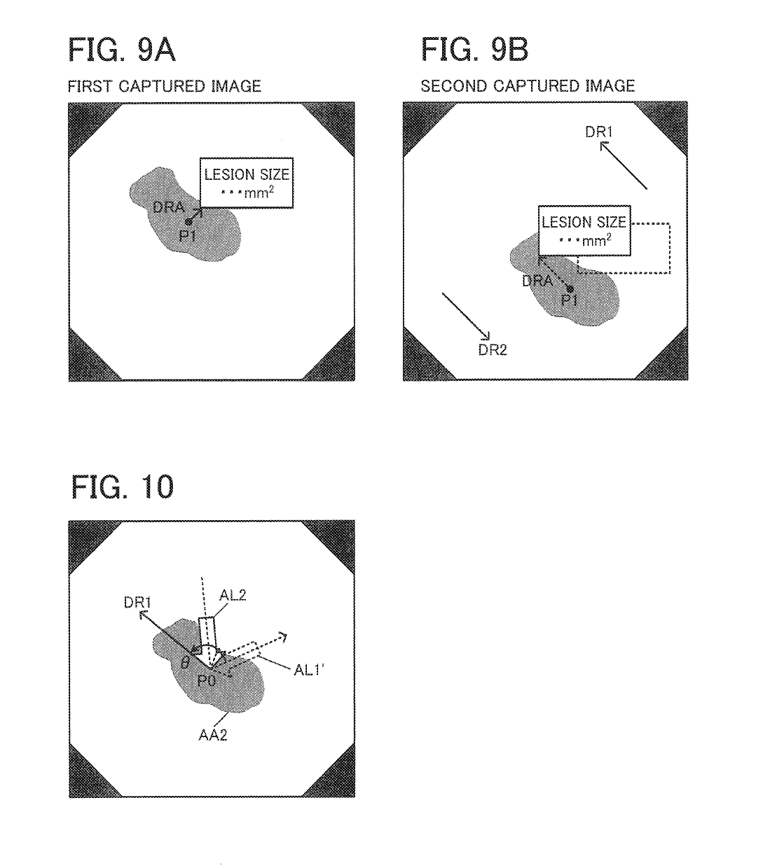

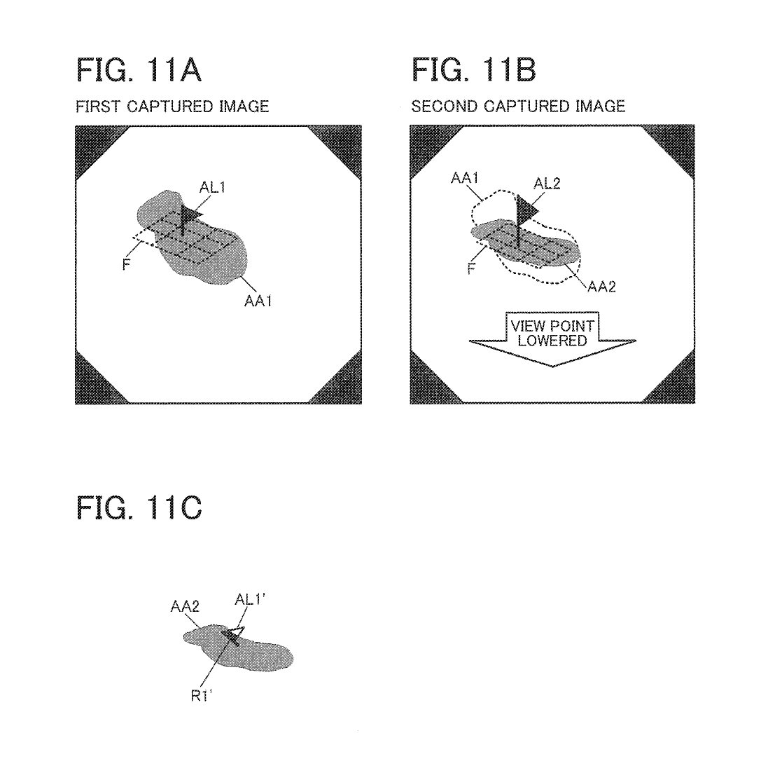

The attention region herein means an region with a relatively higher priority, in terms of observation by the user, than the other regions. In an example where the user is a physician who performs observation for treatment purposes, the attention region is an region, in a captured image, corresponding to a part with mucosa or lesion. In another example where the user is a physician who wants to observe bubbles or feces, the attention region is an region, in a captured image, corresponding to a part with the bubbles or feces. Thus, the attention region may vary depending on a purpose of the user who performs observation, but is an region with a relatively higher priority, in terms of the observation by the user, than the other regions regardless of the purpose. A method for detecting an attention region is described later. The feature quantity is information on characteristics of the pixels, and includes: a pixel value (at least one of R, G, and B values); a luminance value; parallax; hue; and the like. It is a matter of course that the feature quantity is not limited to these, and may further include other various types of information such as edge information (contour information) of the object and shape information on an region defined by the edge. As described above, the alert image is information, displayed on a captured image, for highlighting the attention region. The alert image may be an image with a shape of an arrow as illustrated in FIG. 3A and the like, an image including character information described later with reference to FIG. 9A, an image with a shape of a flag described later with reference to FIG. 11A, or other images. The alert image according to the present embodiment may be any information with which a position or a size of an attention region or a property or the like of the attention region can be emphasized and presented to the user in an easily recognizable manner. Various modifications can be employed for the form of the alert image.

As described above, the first image region is an region, on the captured image, where the alert image is overlaid on the attention region. FIG. 3A illustrates a first captured image in which an attention region AA1 has been detected and on which an alert image AL1 has been displayed in an overlaid manner. In FIG. 3A, the first image region is an region denoted with R1. The first object region is a region of the object within the first image region R1, in the first captured image illustrated in FIG. 3A.

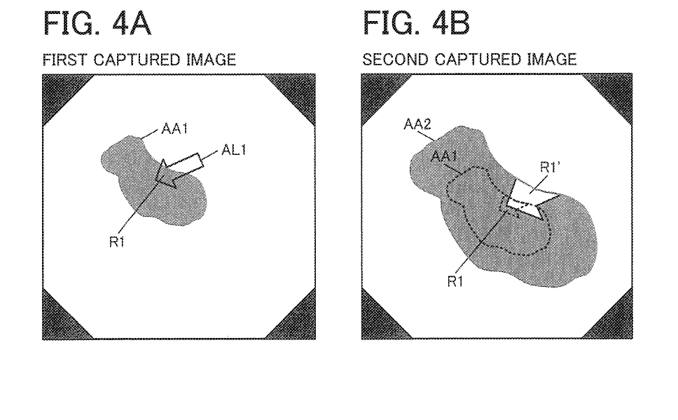

The second image region may be defined primarily based on an region R1′, in an attention region AA2 detected in the second captured image, including a first object region captured. For example, when a relative translational motion between the object and the imaging section 200 occurs during transition between the first captured image and the second captured image as illustrated in FIG. 3B, the region R1′ is an region on the second captured image as a result of the translational motion of R1 as illustrated in FIG. 3B. When zoom-in occurs during the transition between the first captured image and the second captured image as illustrated in FIGS. 4A and 4B, the region R1′ is an region on the second captured image as a result of enlarging R1 as illustrated in FIG. 4B. As described above, the region R1′ is an region of the object, in the captured image, corresponding to (in a narrow sense, matching) the region R1, with the position, the size, and/or the shape on the image not necessarily matching those of the region R1.

The second image region is an region, in the second captured image, where an alert image AL2 is overlaid on the region R1′. When the alert image AL2 is displayed as in FIG. 3C for example, the second image region is an region denoted with R2 in FIG. 3D. The second object region is a region of the object within the second image region R2, in the second captured image illustrated in FIG. 3D.

Thus, the alert image can be controlled in such a manner that the object region (corresponding to the first object region) hidden by the alert image in the first captured image is at least partially unhidden from the alert image in the second captured image. Specifically, the object difficult to observe in the first captured image can be observed in the second captured image, whereby the observation condition can be appropriately improved. This can be achieved with the display control on the alert image based on a motion vector, whereby there is an advantage in that the user needs not to perform a cumbersome operation for controlling the alert image.

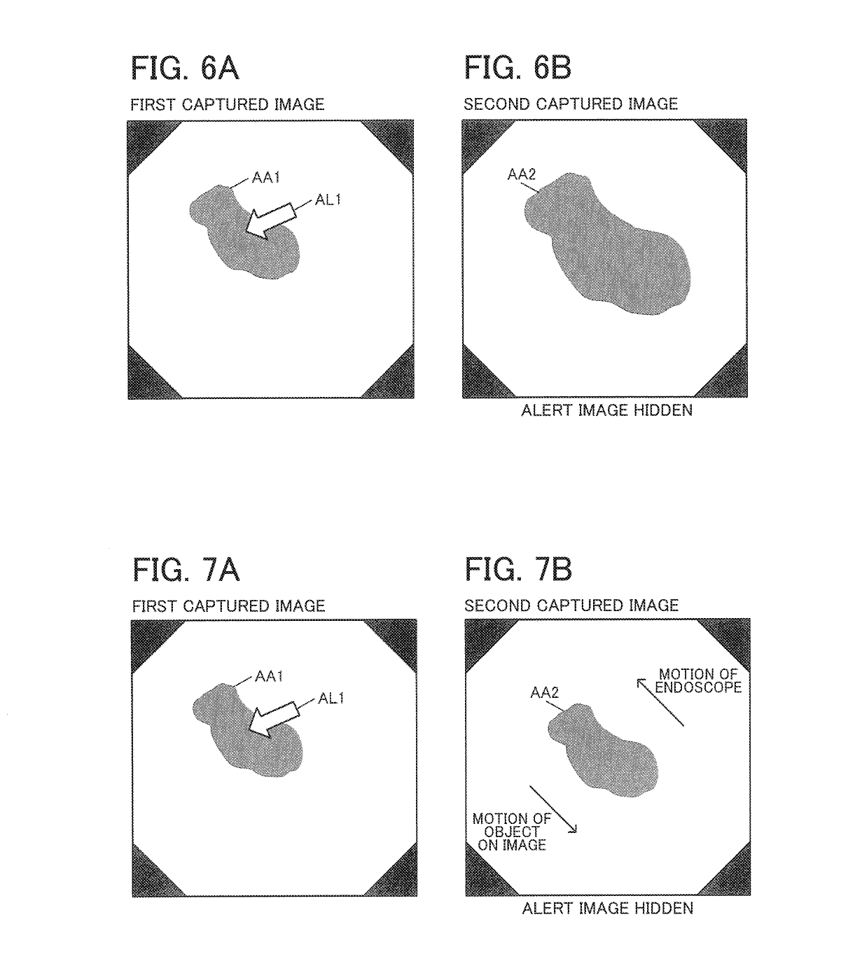

A specific method of performing display control on an alert image in the second captured image for achieving the second object region that is smaller than the first object region is described in detail later with reference to FIG. 6 to FIG. 15.

The description above is based on the sizes (regions) of the first and the second object regions. However, the method according to the present embodiment is not limited to this. For example, the endoscope apparatus according to the present embodiment, may include the image acquisition section 310, the attention region detection section 320, the motion vector estimation section 340, and the display control section 350 described above, the first image region may be an region, in the first captured image, in which the alert image is overlaid on the attention region, the second image region may be an region, in the second captured image, in which the alert image is overlaid on an region corresponding to the first image region, and the display control section 350 may perform display control on the alert image in the second captured image to achieve the second image region that is smaller than the first image region. Specifically, the display control for achieving the second image region that is smaller than the first image region is performed to satisfy a relationship SI2<SI1, where SI2 represents the region of the second image region, and SI1 represents the region of the first image region. Thus, the method according to the present embodiment may include performing display control based on the regions on the captured image.

A specific example of a detection process based on a motion vector and a specific example of the display control of the alert image are described below. The method according to the present invention may involve various combinations between a type of movement detected based on a motion vector and a type of change in the alert image in response to detection of the target movement. Thus, first of all, a basic configuration example is described, and then modifications will be described.

### 2. Basic Embodiment

An endoscope apparatus (endoscope system) according to the present embodiment is described below with reference to FIG. 5. The endoscope apparatus according to the present embodiment includes a rigid scope 100 that is inserted into a body, the imaging section 200 that is connected to the rigid scope 100, a processing section 300, a display section 400, an external I/F section 500, and a light source section 600.

The light source section 600 includes a white light source 610 that emits white light, and a light guide cable 620 that guides the light emitted from the white light source 610 to the rigid scope.

The rigid scope 100 includes a lens system 110 that includes an objective lens, a relay lens, an eyepiece, and the like, and a light guide section 120 that guides the light emitted from the light guide cable 620 to the end of the rigid scope.

The imaging section 200 includes an imaging lens system 240 that forms an image of the light emitted from the lens system 110. The imaging lens system 240 includes a focus lens 220 that adjusts an in-focus object plane position. The imaging section 200 also includes the image sensor 250 that photoelectrically converts the reflected light focused by the imaging lens system 240 to generate an image, a focus lens driver section 230 that drives the focus lens 220, and an auto focus (AF) start/stop button 210 that controls AF start/stop.

For example, the image sensor 250 is a primary color Bayer image sensor in which any one of R, G, and B color filters are disposed in a Bayer array. The image sensor 250 may be any other image sensors such as an image sensor that utilizes a complementary color filter, a stacked image sensor that is designed so that each pixel can receive light having a different wavelength without utilizing a color filter, and a monochrome image sensor that does not utilize a color filter, as long as the object can be captured to obtain an image. The focus lens driver section 230 is implemented by any actuator such as a voice coil motor (VCM), for example.

The processing section 300 includes the image acquisition section 310, the attention region detection section 320, an image storage section (storage section) 330, the motion vector estimation section 340, and the display control section 350 as described above with reference to FIG. 2.

The image acquisition section 310 acquires a captured image obtained by the imaging section 200. The captured image thus obtained is, in a narrow sense, time series (chronological) images. For example, the image acquisition section 310 may be an A/D conversion section that performs processing of converting analog signals sequentially output from the image sensor 250 into a digital image. The image acquisition section 310 (or an unillustrated pre-processing section) may also perform pre-processing on the captured image. Examples of this pre-processing include image processing such as white balance processing and interpolation processing (demosaicing processing).

The attention region detection section 320 detects an attention region in the captured image. The image storage section 330 stores (records) the captured image. The motion vector estimation section 340 estimates a motion vector based on the captured image at a processing target timing and a captured image obtained in the past ((in a narrow sense, obtained at a previous timing) and stored in the image storage section 330. The display control section 350 performs the display control on the alert image based on a result of detecting the attention region and the estimated motion vector. The display control section 350 may perform display control other than that for the alert image. Examples of such display control include image processing such as color conversion processing, grayscale transformation processing, edge enhancement processing, scaling processing, and noise reduction processing. The display control on the alert image is described later in detail.

The display section 400 is a liquid crystal monitor, for example. The display section 400 displays the image sequentially output from the display control section 350.

The processing section 300 (control section) is bidirectionally connected to the external I/F section 500, the image sensor 250, the AF start/stop button 210 and the light source section 600, and exchanges a control signal with these components. The external I/F section 500 is an interface that allows the user to perform an input operation on the endoscope apparatus, for example. The external I/F section 500 includes a setting button for setting the position and the size of the AF region, an adjustment button for adjusting the image processing parameters, and the like.

FIG. 5 illustrates an example of a rigid scope used for laparoscopic surgery or the like. The present embodiment is not limited to the endoscope apparatus with this configuration. The present embodiment may be applied to other endoscope apparatuss such as an upper endoscope and a lower endoscope. The endoscope apparatus is not limited to the configuration illustrated in FIG. 5. The configuration may be modified in various ways with the components partially omitted, or additional components provided. For example, the endoscope apparatus illustrated in FIG. 5 is supposed to perform AF and thus includes the focus lens 220 and the like. Alternatively, the endoscope apparatus according to the present embodiment may have a configuration of not performing AF. In such a configuration, the components for the AF may be omitted. As described below, a zooming operation implemented with the imaging lens system 240 may be performed in the present embodiment. In this configuration, the imaging lens system 240 may include a zoom lens not illustrated in FIG. 5.

Next, processing executed by the attention region detection section 320, the motion vector estimation section 340, and the display control section 350 is described in detail.

Various methods for detecting an attention region, that is, a lesion part in tissue have been proposed. For example, a method according to “Visual SLAM for handheld monocular endoscope” Grasa, Oscar G and Bernal, Ernesto and Casado, Santiago and Gil, Ismael and Montiel, Medical Imaging, Vol. 33, No. 1, p. 135-146, 2014 may be employed, or a shape and a color of an region may be used as disclosed in JP-A-2007-125373. In JP-A-2007-125373, an elliptical shape is extracted from a captured image, and an attention region is detected based on a process of comparing the color in the extracted elliptic shape and the color of a lesion model defined in advance. Alternatively, Narrow band imaging (NBI) may be employed. NBI employs light with a wavelength band smaller than that of basic colors R, G, and B (e.g., B2 (390 nm to 445 nm) or G2 (530 nm to 550 nm)). Thus, a predetermined lesion is displayed with a unique color (for example, reddish brown). Thus, an attention region can also be detected by determining color information or the like of an object, by using narrow band light. The present embodiment may employ a wide variety of other detection methods.

When the attention region detection section 320 detects an attention region, the display control section 350 displays the alert image AL in an overlaid manner at a position on the detected attention region AA, as illustrated in the section A3 in FIG. 1. In this state, the region hidden by the alert image AL cannot be observed. The alert image AL is not limited to the arrow, and may be an image for presenting the type of the detected lesion, details of the patient, and information observed with other modalities (a medical image device or a modality device), with characters, shapes, colors, or the like.

The display control section 350 changes the form of the alert image in such a manner that when an attention region is detected in sequential time series images, an region hidden by the alert image AL in an earlier one of the images can be observed in a later one of the images.

More specifically, the motion vector estimation section 340 estimates a motion vector based on at least one pair of matching points by using a past image stored in the image storage section 330. More specifically, the endoscope apparatus includes the storage section (image storage section 330) that stores captured images, and the motion vector estimation section 340 may detect at least one corresponding pixel (matching point) based on the process of comparing the captured image at the processing timing and a captured image captured before the processing timing and stored in the storage section, and estimate the motion vector based on the corresponding pixel.

Various methods for estimating a motion vector based on matching points in images have been proposed. For example, a method disclosed in JP-A-2009-226072 may be employed. Motion vector estimation is not necessarily based on the motion vector related to the matching points in images. Specifically, a method of estimating a position and a direction of an end of an endoscope based on three-dimensional data acquired in advance, and an estimation method of directly detecting the movement of an endoscope with an external sensor have been known. Thus, the present embodiment may employ a wide variety of motion vector estimation including these methods. The display control section 350 changes a form of the alert image based on the estimated motion vector.

FIG. 6A to FIG. 7B illustrate specific embodiments. The motion vector estimation section 340 estimates a motion vector of at least one matching point around the attention region detected by the attention region detection section 320. The display control section 350 performs control for removing the alert image based on the motion vector or not removing the image. Thus, the display control on an alert image in the second captured image according to the present embodiment may be control for removing the alert image displayed on the first captured image.

As described above, in the present embodiment, the observation condition of an object compromised by the alert image can be improved. Specifically, when the alert image is removed (hidden) in the second captured image, the attention region is not hidden by the alert image in the second captured image. Thus, the second object region that is smaller than the first object region can be achieved, with the second image region and the second object region each having a size (region) of 0.

However, the alert image is for presenting the position as well as detailed information or the like of the attention region to the user, meaning that the amount of information provided to the user decreases when the alert image is removed. For example, when the alert image is removed in a situation where the visibility of the attention region is low, the user might overlook the attention region. Furthermore, detailed information might be removed even when the user wanted to see the information. Thus, before removing the alert image, it is desirable to determine whether or not this removal control has a negative impact.

Thus, in the present embodiment, the observation condition of the user may be estimated based on the motion vector. More specifically, whether or not the user is attempting detailed observation on the target attention region may be estimated. When the user attempting detailed observation cannot observe part of the attention region hidden by the alert image, the user feels a huge stress and might even result in unsatisfactory diagnosis with the lesion overlooked, for example. Thus, the alert image should be removed when the user is estimated to be attempting detailed observation.

For example, it is reasonable to estimate that the user is attempting detailed observation of the attention region when zooming (zoom-in) to the attention region is performed. More specifically, a motion vector related to at least two matching points around a lesion part detected in a past image (first captured image) illustrated in FIG. 6A and around a lesion part detected in a current image (second captured image) illustrated in FIG. 6B is estimated. Then, the user is determined to be performing zooming for the lesion part with the endoscope, when a distance between the two matching points is increasing. Based on this determination result indicating the zooming, the alert image displayed on the first captured image is removed in the second captured image illustrated in FIG. 6B. In a state illustrated in FIG. 6B, illustrating a state corresponding to that in FIG. 4B, the alert image AL2 is hidden and thus is not overlaid on the image region R1′ corresponding to the first object region illustrated in FIG. 4B. Thus, the second image region and the second object region each have an region of 0.

Alternatively, a motion vector may be estimated that is related to at least one matching point around a lesion part detected in a past image as illustrated in FIG. 7A and around a lesion part detected in the current image as illustrated in FIG. 7B. When the motion vector is directed toward the image center, the user may be determined to have noticed the lesion and will start detailed observation. Also in this case, the alert image displayed in the first captured image is removed in the second captured image illustrated in FIG. 7B. Also in a state illustrated in FIG. 7B, illustrating a state corresponding to that in FIG. 3B, the alert image AL2 is hidden and thus is not overlaid on the image region R1′ corresponding to the first object region illustrated in FIG. 3B. Thus, the second image region and the second object region each have an region of 0.

FIG. 7A and FIG. 7B illustrate an example where the attention region is moving toward the image canter through the translational motion. However, this should not be construed in a limiting sense. The alert image may be removed when the attention region is moving toward the image center through rotational motion. The rotational motion may be implemented with the rigid scope 100 (portion to be inserted) of the endoscope apparatus rotating about the optical axis, for example.

In the present embodiment, the display control section 350 may perform the display control on the alert image in the second captured image, in such a manner that S2<S1 holds true, where S1 represents an region of the first object region and S2 represents an region of the second object region. Thus, achieving the second object region that is smaller than the first object region may include setting the regions S1 and S2 of the object regions and satisfying the relationship S2<S1. As used herein, the region of each object region may be the surface region of an region of the object overlaid on the corresponding region, or may be the region of an region (object plane) as a result of projecting the object onto a predetermined plane (for example, a plane orthodontal to the optical axis direction of the imaging section 200). In any cases, the object region according to the present embodiment represents the size of the object in regionl space, and thus does not necessarily match the size (region) on the image. For example, as described above with reference to FIG. 4A and FIG. 4B, the region of one object region on an image changes when the distance between the object and the imaging section 200 and an optical system condition such as zoom ratio changes.

The display control section 350 may perform the display control on the alert image in the second captured image to achieve the second object region that is smaller than the first object region, when the imaging section 200 is determined to have made zooming on the object, during the transition between the first captured image and the second captured image, based on the motion vector.

Alternatively, the display control section 350 may perform the display control on the alert image in the second captured image to achieve the second object region that is smaller than the first object region, when the imaging section 200 is determined to have made at least one of a translational motion and a rotational motion relative to the object, during the transition between the first captured image and the second captured image, based on the motion vector.

Thus, whether or not the zooming or a translational or rotational motion has occurred can be determined based on the motion vector, and the display control on an alert image can be performed based on a result of the determination. Thus, the user only needs to perform an operation involving zooming or a translational or rotational motion. For example, when the imaging lens system 240 includes a zoom lens, the zooming can be implemented by controlling the zoom lens (controlling zoom ratio). The zooming can also be implemented by reducing the distance between the imaging section 200 and the object. The translational motion may be implemented with the imaging section 200 (rigid scope 100) moved in a direction crossing (in a narrow sense, a direction orthogonal to) the optical axis. The rotational motion may be implemented with the imaging section (rigid scope 100) rotated about the optical axis. These operations are naturally performed when an object is observed with the endoscope apparatus. For example, these operations are performed for positioning to find an attention region and achieve a better view of the attention region found. All things considered, the display mode of the alert image can be changed without requiring dedicated operations for the change, and thus can be changed through the operation naturally involved in the endoscope observation.

Such processing may involve control performed by the display control section 350 for hiding the alert image in the second captured image. More specifically, as described above, the display control section 350 may perform the control for hiding the alert image in the second captured image, when the zooming is determined to have been performed for the attention region, during the transition between the first captured image and the second captured image, based on the motion vector. Alternatively, the display control section 350 may perform the control for hiding the alert image in the second captured image, when the attention region is determined have moved toward the captured image center, during the transition between the first captured image and the second captured image, based on the motion vector.

As described above, the motion vector according to the present embodiment may be any information indicating a movement of an object on the captured image, and thus is not limited to information obtained from the image. For example, the rigid scope 100 may be provided with a motion sensor of a certain kind (for example, an acceleration sensor or a gyroscope sensor), and the motion vector according to the present embodiment may be obtained based on sensor information from the motion sensor. In the configuration of implementing the zooming by controlling the zoom lens, whether or not the zooming is performed may be determined based on the motion vector obtained based on control information on the zoom lens. Furthermore, the motion vector may be obtained with a combination of a plurality of methods. Specifically, the motion vector may be obtained based on both sensor information and image information.

Thus, the alert image can be removed when the zooming to the attention region, or the movement of the attention region toward the image center is detected. Thus, whether or not the user is determined to be attempting to observe the attention region may be determined, and the alert image can be removed when the user is determined to be attempting to observe the attention region. When the user is attempting to observe the attention region, the alert image hiding attention region should have a huge negative impact. Thus, hiding the alert image is highly effective. When detailed observation is to be performed, importance of the arrow indicating a position, detailed information, or the like is relatively low. Thus, removing the alert image is less likely to be disadvantageous. For example, the user paying attention to the attention region is less likely to miss the position of the attention region, whereby the arrow may be removed. The user performing the zooming or the like is supposed to visually check the object in the attention region, and thus is less likely to be required to also see the detailed alert image including the character information or the like.

The endoscope according to the present embodiment may include a processor and a memory. The processor may be a central processing unit (CPU), for example. Note that the processor is not limited to a CPU. Various other processors such as a graphics processing unit (GPU) or a digital signal processor (DSP) may also be used. The processor may be a hardware circuit that includes an application-specific integrated circuit (ASIC). The memory stores a computer-readable instruction. Each section of the endoscope apparatus according to the present embodiment is implemented by causing the processor to execute the instruction. The memory may be a semiconductor memory (e.g., SRAM or DRAM), a register, a hard disk, or the like. The instruction may be an instruction included in an instruction set that is included in a program, or may be an instruction that causes a hardware circuit included in the processor to operate.

As described above, the present embodiment enables the operator to perform control for changing, displaying, or hiding the mark (alert image) provided to the attention region, by moving the imaging section 200 (rigid scope 100). Thus, the operator who wants to move the mark provided to the attention region can perform the control through a natural operation, without requiring a special switch. In this process, the mark can be hidden when the operator zooms into the attention region or moves the attention region toward the center. Thus, the operator who wants to move the mark provided to the attention region can perform the control through a natural operation, without requiring a special switch.

### 3. Modification

The determination based on the motion vector and the display control on an alert image according to the present embodiment are not limited to those described above. Some modifications are described below.

### 3.1 Rotational Display

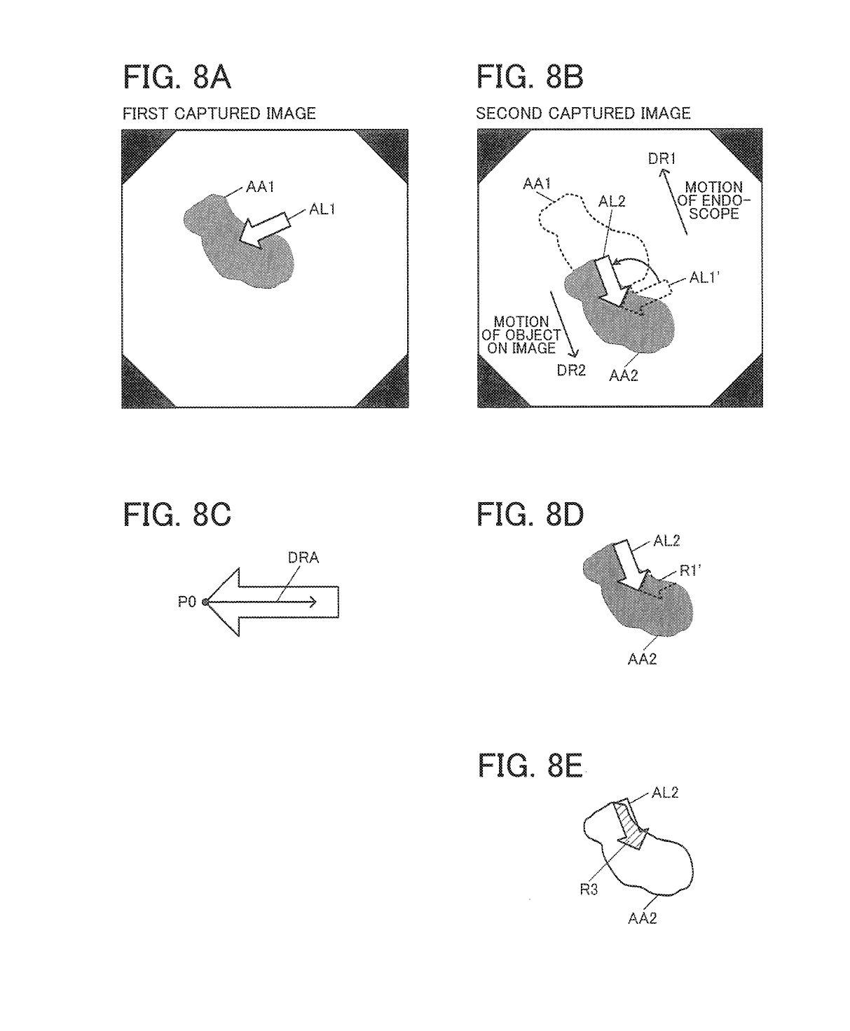

As illustrated in FIG. 8A and FIG. 8B, the display control section 350 may perform control for rotating the alert image on the first captured image and displaying the resultant image on the second captured image based on the motion vector. This will be described in detail below. Compared with the first captured image illustrated in FIG. 8A, the second captured image illustrated in FIG. 8B has the attention region positioned farther in a lower right direction (DR2) due to a relative movement of the imaging section 200 (rigid scope 100) in an upper left direction (DR1). The directions DR1 and DR2 are opposite to each other.

Here, a motion vector in DR1 or DR2 is detected. The description is given below under an assumption that the motion vector is obtained through image processing on the captured image, and the motion vector in DR2 is detected.

FIG. 8B illustrates an alert image AL1′ displayed on the second captured image without ruining the relative relationship between the attention region AA1 and the alert image AL1 in the first captured image. For example, the alert image AL1′ can be positioned on the second captured image without ruining the relative relationship, by being disposed with an arrow serving as the alert image having an end position staying at a predetermined position (for example, the position at the center, a gravity center, or the like) of the attention region, and having an orientation (an angle and a direction) unchanged.

In the present embodiment, the alert image AL2 displayed on the second captured image is determined with AL1′ before the rotation (starting point of the rotation) rotated based on the direction DR2 of the estimated motion vector. For example, the rotation may be performed about a predetermined position of the alert image in such a manner that the direction of the alert image matches the direction DR1 opposite to the direction DR2 of the motion vector.

For example, when the alert image is an arrow image including a shaft and an arrow head provided on one end of the shaft, the predetermined position of the alert image as the rotational center may be a distal end (P0) of the arrow head as illustrated in FIG. 8C. The direction of the alert image may be a direction (DRA) from the distal end P0 of the arrow head toward an end of the shaft without the arrow head. In this case, the alert image AL2 is obtained by performing the rotation about P0 in such a manner that DRA matches DR1 in FIG. 8B.

Thus, the first captured image and the second captured image have different positions of the alert image relative to the attention region. Thus, at least a part of the image region R1′ corresponding to the first object region is not overlaid on the alert image AL2 in the second captured image as illustrated in FIG. 8D. As a result, the object difficult to observe in the first captured image can be easily observed in the second captured image. Specifically, in the examples illustrated in FIG. 8B and FIG. 8D, AL2 is not overlaid on R1′ (the second image region and the second object region each have a size=0). It is a matter of course that AL2 might be overlaid on R1′, that is, the attention region might be not be visible in the first captured image and in the second captured image, depending on a relationship among P0, DRA, and DR1. Still, the method illustrated in FIG. 8A to FIG. 8D features the rotational motion of the alert image, achieving a relative relationship between the attention region AA2 and the alert image AL2 in the second captured image different from the relative relationship between the attention region AA1 and the alert image AL1 in the first captured image. All things considered, the second object region having a smaller region than the first object region can be achieved, whereby the observation condition can be improved with at least a part of an region unable to be observed in the first captured image being observable in the second captured image.

As is apparent in FIG. 8B illustrating the present modification, the alert image is not removed in the second captured image. Thus, the alert image AL2 may be overlaid on the attention region AA2 in the second captured image, rendering observation of an region (R3 in FIG. 8E) difficult. Under a certain condition, (the size of the object region R3)>(the size of the first object region R1) might hold true. Still, the method according to the present embodiment is directed to display control enabling the object unable to be observed before a movement operation by the user (in the first captured image) to be more easily observed after the movement operation (in the second captured image). Thus, hiding of the object that has been observable by the alert image as a result of the display control is tolerated because it would not be critical. Specifically, even when the region (R3) as a part of the attention region in the second captured image becomes unable to be observed, further zooming or the translational or rotational motion caused by the user triggers the display control for improving the observation condition for the partial region in the next captured image (third captured image).

The alert image as a target of the display control according to the present modification is not limited to the arrow. For example, the following modification may be employed. Specifically, an attention region provided with an alert image including characters and the like displayed on the DRA side relative to the reference position in the first captured image as illustrated in FIG. 9A may be moved in the direction DR2 in the second captured image as illustrated in FIG. 9B. In such a case, the alert image including characters and the like may be displayed on the DR1 side relative to the reference position in the second captured image.

The motion vector may be rotated in the direction DR1, by the rotation amount corresponding to the amount of movement (the size of the motion vector). For example, when the movement amount is larger than a predetermined threshold value Mth, the rotation may be performed to make DRA match DR1 as in FIG. 8A and FIG. 8B. When the movement amount is M (<Mth), the rotation amount may be obtained by θ×M/Mth where θ represents an angle between DRA and DR1 before the rotation. For example, with the movement amount M=Mth/2, the rotation amount of the alert image is θ/2, whereby the alert image AL2 is displayed at the position illustrated in FIG. 10. In this manner, the movement amount (rotation amount) of the rotational motion of the alert image can be controlled based on the size (movement amount) of the motion vector.

As described above, in the present modification, when the attention region has made the translational motion in the first direction (corresponding to DR2 in FIG. 8B and the like) during the transition between the first captured image and the second captured image, based on the motion vector, the display control section 350 performs control in such a manner that the alert image makes the rotational motion in the direction (DR1) opposite to the first direction, with the attention region in the second captured image as a reference, and the resultant image is displayed on the second captured image.

Thus, the alert image (mark) provided to the attention region can be rotated in accordance with the movement of the imaging section 200 by the user, whereby when the operator wants to move the alert image, the control can be performed through a natural operation without requiring a special switch. In this process, the rotational direction is set based on the direction of the motion vector so that the alert image moves based on the physical law in the real space, whereby an intuitive operation can be achieved. The control illustrated in FIG. 8A and FIG. 8B can be more easily understood with an example where an object moves while holding a pole with a flag. When the object moves in a predetermined direction while holding the flag, a material (cloth, paper, or the like) attached to a distal end of the pole trails in a direction opposite to the direction of the movement by receiving an air flow in the direction opposite to the movement direction.

Also in the example illustrated in FIG. 8A and FIG. 8B, the attention region moves in the direction DR2, and the alert image rotates to be disposed at a position on the side of the direction DR1 opposite to the movement direction. The alert image can also be regarded as trying to stay stationary despite the movement of the attention region in the direction DR2. An object being dragged in the direction opposite to the movement direction (trying to stay), as in the example of the flag described above and an example involving large inertia, is a common physical phenomenon. Thus, with the alert image moving in a similar manner in the captured image, the user can intuitively control the alert image. The rotation amount may be further associated with the size of the motion vector so that the control conforming to the movement of the object in the real space can be achieved, whereby more user-friendly control can be implemented. For example, the alert image can be controlled in accordance with a basic principal including regiondily understood phenomenon that a slight movement of the flag pole only results in a small fluttering of the cloth.

In the description above, the relative translational motion between the imaging section 200 and the object is detected based on the motion vector. Alternatively, control for rotating the alert image when the relative rotational motion between the imaging section 200 and the object is detected, and displaying the resultant image may be performed. Also in this configuration, the rotational direction and the rotation amount of the alert image may be set based on the direction and the size of the motion vector.

In the modification described above, the alert image continues to be displayed in the second captured image with the displayed position and orientation controlled based on the motion vector. The movement detected based on the motion vector is not limited to the movement of the attention region toward the image center. For example, the concept of the present modification well includes an operation of moving the attention region toward an image edge portion, for changing the relative position and orientation of the alert image relative to the attention region (for improving the observation condition).

### 3.2 Pan/Tilt

In the description above, the relative movement between the imaging section 200 and an object includes zooming, a translational motion, and a rotational motion (in a narrow sense, rotation about the optical axis corresponding to roll). However, the relative movement is not limited to these. For example, three-orthogonal axes may be defined with the optical axis of the imaging section 200 and two axes orthogonal to the optical axis, and movements each representing rotation about a corresponding one of the two axes orthogonal to the optical axis may be detected based on a motion vector, to be used for the display control. Specifically, these movements correspond to pan and tilt.

In the present modification, the endoscope apparatus (in a narrow sense, the processing section 300) may include an attention region normal line estimation section not illustrated in FIG. 5 or the like. The attention region normal line estimation section estimates a normal direction of a three-dimensional tangent plane relative to a line-of-sight direction of the endoscope around the attention region based on the matching points and the motion vector estimated by the motion vector estimation section 340. Various methods for estimating the normal direction of the three-dimensional tangent plane relative to the line-of-sight direction of the endoscope have been proposed. For example, a method disclosed in “Towards Automatic Polyp Detection with a Polyp Appearance Model” Jorge Bernal, F. Javier Sanchez, & Fernando Vilarino, Pattern Recognition, 45 (9), 3166-3182 may be employed. Furthermore, the processing for estimating the normal direction executed by the attention region normal line estimation section according to the present embodiment may employ a wide variety of methods other than these.

The display control section 350 changes the form of the alert image based on the estimated normal direction and presents the resultant image. This operation is described more in detail with reference to FIG. 11A and FIG. 11B. FIG. 11A illustrates a first captured image in which a tangent plane F corresponding to an attention region AA has been estimated, and an alert image with a shape of a flag is displayed to stand in the normal direction of the tangent plant F.

In this example, the first image region and the first object region difficult to observe correspond to an region behind the flag. When the user moves the imaging section 200 (rigid scope 100) toward the tangent plane F as in the second captured image illustrated in FIG. 11B to observe the region behind the flag, the normal direction changes. In the present modification, the form of the alert image having a shape of the flag changes based on the change in the normal direction, so that the region behind the flag can be observed as in FIG. 11B. In this case, the image region R1′, in the second captured image, corresponding to the first object region is as illustrated in FIG. 11C. Thus, the second image region R2 may be regarded as the region where R1′ is overlaid on AL2 in FIG. 11B. Apparently, R2 is at least a part of R1′. Thus, the present modification can also achieve the second object region that is smaller than the first object region.

In the present modification described above, the display control section 350 performs the display control on an alert image in the second captured image to achieve the second object region that is smaller than the first object region, when movement involving a change in an angle between the optical axis direction of the imaging section 200 and the normal direction of the object is determined to have been performed, during the transition between the first captured image and the second captured image, based on the motion vector.

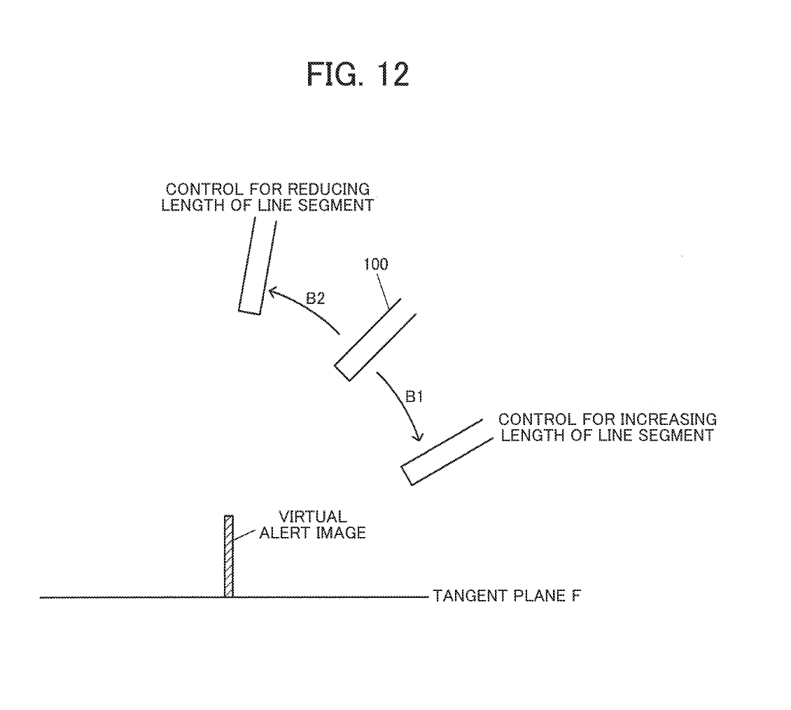

More specifically, the alert image may be regarded as a virtual object on the three-dimensional space, and an image obtained by observing the alert image from a virtual view point determined based on the position of the imaging section 200 may be displayed on the second captured image. A method of arranging an object in a virtual three-dimensional space and generating a two-dimensional image obtained by observing the object from a predetermined view point has been widely known in a field of computer graphics (CG) or the like, and thus the detail description thereof is omitted. For the alert image having a shape of a flag as in FIG. 11B, the display control section 350 may perform a simple calculation instead of an intricate calculation for projecting a two-dimensional image of a three-dimensional object. For example, as illustrated in FIG. 12, the display control section 350 may perform display control of estimating a normal direction of a plane of an attention region based on a motion vector, and changing the length of a line segment in the normal direction. When the imaging section 200 (rigid scope 100) is operated to rotate toward the tangent plane, that is, when the imaging section 200 is operated to move in such a direction to have the optical axis included in the tangent plane as indicated by B1 in FIG. 12, the length of the line segment in the normal direction may be increased from that before the movement as illustrated in FIG. 11B. When the optical axis of the imaging section 200 moves toward the normal direction of the tangent plane as indicated by B2 in FIG. 12, the length of the line segment in the normal direction may be reduced from that before the movement.

In this manner, the alert image (mark) provided to the attention region can be changed in accordance with the movement of the imaging section 200 by the operator. Thus, the operator who wants to move the alert image can perform the control through a natural operation without requiring a special switch. In the present modification, the alert image is displayed as if it is an actual object in three-dimensional space, or such a display mode can be easily implemented. Thus, the user can easily recognize how to move the imaging section 200 to observe an object hidden by the alert image (behind the alert image). All things considered, the observation condition of the attention region can be improved through an intuitively recognizable operation.

The display control on an alert image performed in such a manner that the shape of the alert image changed when a pan/tilt operation is detected is described above. However, this should not be construed in a limiting sense. For example, the alert image may be removed or may make the rotational motion to be displayed when the pan/tilt operation is detected. In such a case, whether or not to perform the removal, as well as the direction and the amount of the rotational motion may be determined based on the direction or the size of the motion vector.

### 3.3 Size Change

In the description above, the change in the alert image includes removal, rotational motion, and shape change (change in a projection direction in which a two-dimensional image of a virtual three-dimensional object is projected). The change may further include other types of changes. For example, the display control section 350 may perform control for changing the size of the alert image in the first captured image based on the motion vector and displaying the resultant image on the second captured image.

For example, the display control section 350 performs control for reducing the size of the alert image in the first captured image and displaying the resultant image in the second captured image, when the zooming is determined to have been performed for the object with the imaging section 200, during the transition between the first captured image and the second captured image, based on the motion vector.

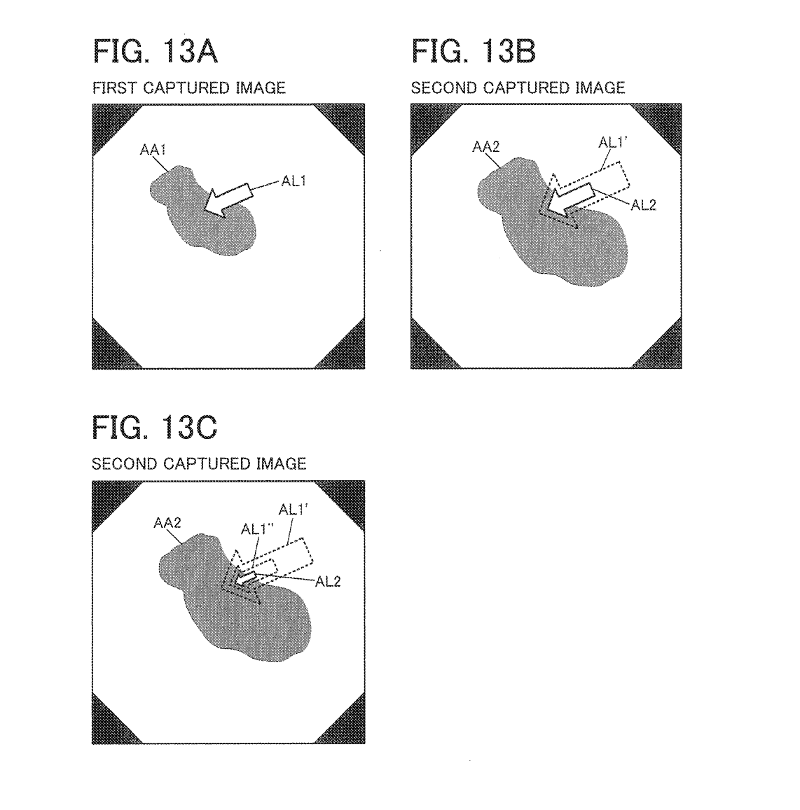

FIG. 13A to FIG. 13C illustrate a specific example. FIG. 13A illustrates a first captured image as in FIG. 4A and the like. A second captured image is obtained as a result of zooming as illustrated in FIG. 13B, and the image region R1′ corresponding to the first object region is a result of enlarging the first image region R1, as described above with reference to FIG. 4B. Thus, when the alert image displayed in the second captured image has a size substantially the same as that in the first captured image, the alert image AL2 is only partially overlaid on R1′ as illustrated in FIG. 13B, whereby a second object region smaller than a first object region can be achieved.

In the present modification, the size of the alert image is reduced as described above, whereby the observation condition can be improved from that in the configuration where the size of the alert image remains the same. More specifically, as illustrated in FIG. 13C, the alert image AL2 has the size smaller than that of the alert image AL1 in the first captured image (corresponding to AL1″ in FIG. 13C). Thus, an region overlaid on R1′ can further be reduced from that in FIG. 13B, whereby the observation condition can further be improved. The user who has performed the zooming is expected to be attempting to observe a predetermined object in detail. Thus, the size reduction of the alert image should less likely to have a negative impact.

Although the zooming (zoom-in in particular) is described above, the movement for changing the size of the alert image is not limited to this. More specifically, the size of the alert image may be changed in a case where the relative translational or rotational motion occurs between the imaging section 200 and the object, when a pan/tilt operation is performed, or in the other like cases. Although not described above, the magnification for changing the size may be determined based on the size of the motion vector and the like.

### 3.4 Plurality of Alert Images

In the example described above, a single alert image is displayed for a single attention region. However, this should not be construed in a limiting sense, and a plurality of alert images may be displayed for a single attention region.

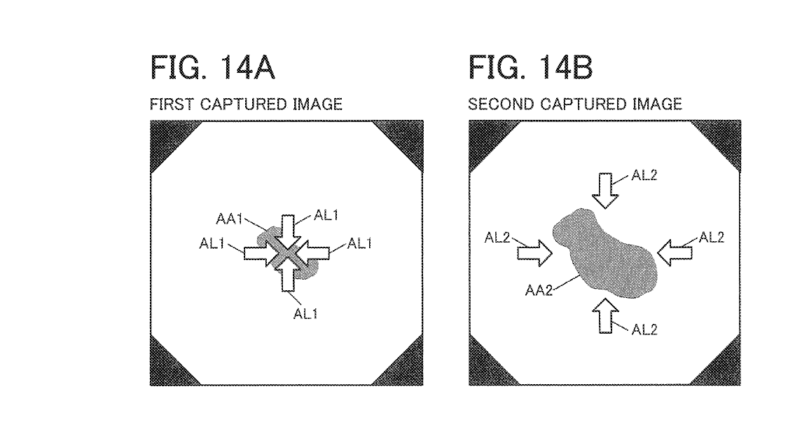

This example is illustrated in detail in FIG. 14A and FIG. 14B. In FIG. 14A, four alert images (arrows) are displayed for a single attention region. For example, the alert images may be displayed to surround the attention region (with the center of a distal end portion of each of the four arrows disposed at a predetermined position on the attention region).

The display control section 350 may perform control for causing the alert image to make a translational motion toward an edge portion of the captured image, and displaying the resultant image on the second captured image, when the zooming is determined to have been performed for the attention region, during the transition between the first captured image and the second captured image, based on the motion vector, as illustrated in FIG. 14B.

Thus, with this display mode, the position indicated by a plurality of alert images is easily recognizable before the zooming (in the first captured image). Thus, easy recognition of the position of the attention region or the other like effect can be achieved. Furthermore, the alert image makes a relative movement toward the edge portion of the captured image as a result of the zoom-in (in the second captured image). Thus, the observation condition can be improved while maintaining the displaying of the plurality of alert images. The movement toward the edge portion may be achieved with display control for setting a reference position of the alert image (such as a distal end of the arrow) to be closer to an edge (end) of the captured image than the position in the first captured image.

Although an example with a plurality of alert images is described above, the display control for causing an alert image to make a translational motion may be performed also when a single alert image described above is provided. Thus, the display control section 350 may perform control for causing the alert image in the first captured image to make the translational motion based on the motion vector, and displaying the resultant image on the second captured image.

In this process, the direction of the translational motion is not limited to that toward an edge portion, and may be other directions. More specifically, the direction and the amount of the movement of the alert image as a result of the translational motion may be determined based on the direction and the size of the estimated motion vector. The operation associated with the control for causing the alert image to make the translational motion is not limited to the zooming, and the control may be associated with the relative translational motion or the rotational motion (roll) between the imaging section 200 and the object, pan/tilt, or the like.

### 3.5 Multistage Processing

In the example described above, the display control on an alert image in the second captured image is performed based on a result of estimating a motion vector between the first captured image and the second captured image. However, this should not be construed in a limiting sense, and the display control may be performed based on captured images acquired at three or more timings.

For example, the display control section 350 may perform control for displaying an alert image having an region achieving a second object region smaller than a first object region on the second captured image in an overlaid manner, when at least one of zooming for the attention region, the translational motion of the imaging section 200 relative to the object, rotational motion of the imaging section 200 relative to the object, and a movement involving a change in an angle between the optical axis direction of the imaging section 200 and the normal direction of the object is determined to have occurred during the transition between the first captured image and the second captured image, based on a motion vector. The display control section 350 may perform control for hiding the alert image in a third captured image, when at least one of the zooming, the translational motion, the rotational motion, and the movement of changing the angle is determined to have occurred during the transition between the second captured image and the third captured image.

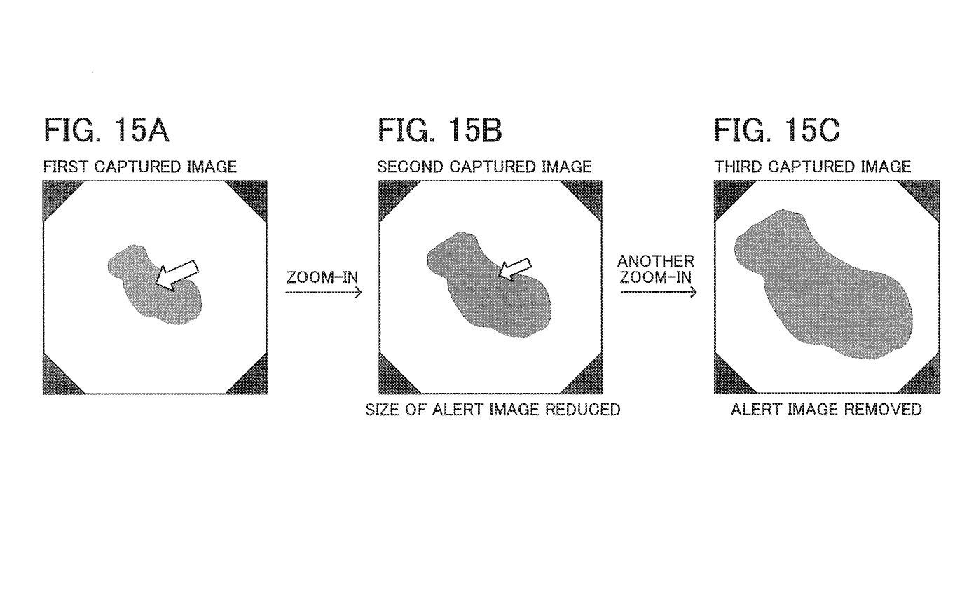

FIG. 15A to FIG. 15C illustrate a flow of the display control in detail. FIG. 15A illustrates the first captured image, FIG. 15B illustrates the second captured image, and FIG. 15C illustrates the third captured image. As described above, the second captured image is acquired later in time than the first captured image (in a narrow sense, at a subsequent timing), and the third captured image is acquired later in time than the second captured image (in a narrow sense, at a subsequent timing). FIG. 15B illustrates a result of display control for reducing the size of the alert image for improving the observation condition, due to zoom-in. FIG. 15C illustrates a result of display control of removing the alert image for improving the observation condition, due to another zoom-in.

In this manner, the display control for improving the observation condition can be performed in multiple stages. As described above, removing the alert image is less likely to have a negative impact when the user wants to observe the attention region in detail. Still, a zoom-in operation or the like performed at a predetermined timing might be an erroneous operation or the like, and thus might be performed even when the user has no intention to observe the attention region in detail. In such a case, removing the alert image might do have a negative impact.

Thus, in the present modification, when the zoom-in is detected once, display control on an alert image different from the removing (such as a translational motion, a rotational motion, and change in shape or size) is performed as a first stage process, instead of immediately removing the alert image. As a result, the alert image continues to be displayed with a different display mode, and thus the process is less likely to have a negative impact for the user who wants to see the alert image. When the zoom-in is further performed in this state, it is reasonable to determine that the user is highly likely to be attempting to observe the attention region in detail. Thus, a second stage process is performed to remove the alert image. With the multistage processing as described above, the display control on an alert image conflicting with the user's intention is less likely to be performed. FIG. 15A to FIG. 15C illustrate an example involving zooming. However, this should not be construed in a limiting sense, and other types of movement may be detected. Furthermore, the first stage and the second stage for detection of the same type of movement should not be construed in a limiting sense. For example, a modification in which zooming is detected in the second captured image and the translational motion of the attention region toward the captured image center is detected in the third captured image may be employed.

Although the present embodiment has been described in detail above, those skilled in the art will readily appreciate that many modifications are possible in the embodiments without materially departing from the novel teachings and advantages of the invention. Accordingly, all such modifications are intended to be included within scope of the invention. Any term cited with a different term having a broader meaning or the same meaning at least once in the specification and the drawings can be replaced by the different term in any place in the specification and the drawings. The configurations and the operations of the endoscope apparatus, and the like are not limited to those described above in connection with the embodiments. Various modifications and variations may be made of those described above in connection with the embodiments. The various embodiments described above are not limited to independent implementation, and a plurality of embodiments may be freely combined.

What is claimed is:

1. An endoscope apparatus comprising:
a processor comprising hardware,
the processor being configured to implement:
an image acquisition process that acquires a captured image, the captured image being an image of an object obtained by an imaging section;
an attention region detection process that detects an attention region based on a feature quantity of pixels in the captured image;
a motion vector estimation process that estimates a motion vector in at least a part of the captured image; and
a display control process that displays an alert image on the captured image in an overlaid manner based on the attention region and the motion vector, the alert image highlighting the attention region,
wherein a first image region is defined as an region, in a first captured image, where the alert image is overlaid on the attention region, and a first object region is defined as an region, on the object, corresponding to the first image region,
wherein a second image region is defined as an region, in a second captured image, where the alert image is overlaid on an image region corresponding to the first object region, and a second object region is defined as an region, on the object, corresponding to the second image region, and
wherein the processor implements the display control process that performs display control on the alert image in the second captured image to achieve the second object region that is smaller than the first object region.

2. The endoscope apparatus as defined in claim 1,
wherein when the imaging section is determined to have made zooming on the object, during transition between the first captured image and the second captured image, based on the motion vector, the processor implements the display control process that performs the display control on the alert image in the second captured image to achieve the second object region that is smaller than the first object region.

3. The endoscope apparatus as defined in claim 1,
wherein when the imaging section is determined to have made at least one of a translational motion and a rotational motion relative to the object, during transition between the first captured image and the second captured image, based on the motion vector, the processor implements the display control process that performs the display control on the alert image in the second captured image to achieve the second object region that is smaller than the first object region.

4. The endoscope apparatus as defined in claim 1,
wherein when movement involving a change in an angle between an optical axis direction of the imaging section and a normal direction of the object is determined to have occurred, during transition between the first captured image and the second captured image, based on the motion vector, the processor implements the display control process that performs the display control on the alert image in the second captured image to achieve the second object region that is smaller than the first object region.

5. The endoscope apparatus as defined in claim 2,
wherein the processor implements the display control process that performs control for hiding the alert image in the second captured image.

6. The endoscope apparatus as defined in claim 5,
wherein when zoom-in to the attention region is determined to have been performed, during the transition between the first captured image and the second captured image, based on the motion vector, the processor implements the display control process that performs the control for hiding the alert image in the second captured image.

7. The endoscope apparatus as defined in claim 5,
wherein when the attention region is determined to have moved toward a center portion of the captured image, during the transition between the first captured image and the second captured image, based on the motion vector, the processor implements the display control process that performs the control for hiding the alert image in the second captured image.

8. The endoscope apparatus as defined in claim 2,
wherein the processor implements the display control process that performs control for causing the alert image in the first captured image to make a rotational motion based on the motion vector, and displaying a resultant image on the second captured image.

9. The endoscope apparatus as defined in claim 8,
wherein when the attention region is determined to have made a translational motion in a first direction, during the transition between the first captured image and the second captured image, based on the motion vector, the processor implements the display control process that performs control for causing the alert image to make a rotational motion in a direction opposite to the first direction of the attention region in the second captured image, and displaying a resultant image on the second captured image.

10. The endoscope apparatus as defined in claim 2,
wherein the processor implements the display control process that performs control for causing the alert image to make a translational motion in the first captured image based on the motion vector and displaying a resultant image on the second captured image.

11. The endoscope apparatus as defined in claim 10,
wherein when zoom-in to the attention region is determined to have been performed, during the transition between the first captured image and the second captured mage, based on the motion vector, the processor implements the display control process that performs control for causing the alert image to make a translational motion in a direction toward an edge portion of the captured image and displaying a resultant image on the second captured image.

12. The endoscope apparatus as defined in claim 2,
wherein the processor implements the display control process that performs control for changing a size of the alert image in the first captured image based on the motion vector and displaying a resultant image on the second captured image.

13. The endoscope apparatus as defined in claim 2,
wherein when the imaging section is determined to have made zoom-in to the object, during transition between the first captured image and the second captured image, based on the motion vector, the processor implements the display control process that performs control for reducing a size of the alert image in the first captured image, and displaying a resultant image on the second captured image.

14. The endoscope apparatus as defined in claim 1,
wherein when at least one of zooming to the attention region, a translational motion of the imaging section relative to the object, a rotational motion of the imaging section relative to the object, and movement involving a change in an angle between an optical axis direction of the imaging section and a normal direction of the object is determined to have occurred, during transition between the first captured image and the second captured image, based on the motion vector, the processor implements the display control process that performs control for displaying the alert image, to achieve the second object region that is smaller than the first object region, on the second captured image in an overlaid manner, and
wherein when at least one of the zooming, the translational motion, the rotational motion, and the movement involving the change in the angle is determined to have occurred between the second captured image and a third captured image, the processor implements the display control process that performs control for hiding the alert image in the third captured image.

15. The endoscope apparatus as defined in claim 1,
further comprising a memory that stores the captured image,
wherein the processor implements the motion vector estimation process that detects at least one corresponding pixel based on a process of comparing between the captured image acquired at a processing timing and a captured image acquired before the processing timing stored in the memory, and estimates the motion vector based on the corresponding pixel.

16. A method for operating an endoscope apparatus comprising:
performing processing to acquire a captured image, the captured image being an image of an object obtained by an imaging section;
detecting an attention region based on a feature quantity of pixels in the captured image;
estimating a motion vector in at least a part of the captured image; and
performing display control to display an alert image on the captured image in an overlaid manner based on the attention region and the motion vector, the alert image highlighting the attention region,
wherein a first image region is defined as an region, in a first captured image, where the alert image is overlaid on the attention region, and a first object region is defined as an region, on the object, corresponding to the first image region,
wherein a second image region is defined as an region, in a second captured image, where the alert image is overlaid on an image region corresponding to the first object region, and a second object region is defined as an region, on the object, corresponding to the second image region, and
wherein in the display control, display control is performed on the alert image in the second captured image to achieve the second object region that is smaller than the first object region.
